(19) United States
(12) Patent Application Publication
(10) Pub. No.: US 2013/0255713 A1
(43) Pub. Date: Oct. 3, 2013

(54) Laminate having improved water retention characteristics

(71) Applicants: Iris Schnitzler, Bonn (DE); Christian Hausen, St. Katharinen (DE); Tobias Kleudgen, Ettringen (DE); Christina Klein, Mayen (DE)

(72) Inventors: Iris Schnitzler, Bonn (DE); Christian Hausen, St. Katharinen (DE); Tobias Kleudgen, Ettringen (DE); Christina Klein, Mayen (DE)

(21) Appl. No.: 13/878,843

(22) PCT Filed: Oct. 6, 2011
(86) PCT No.: PCT/EP2011/004983

§ 371 Date: Apr. 24, 2013

(30) Foreign Application Priority Data
Oct. 15, 2010 (DE) 102010048408.3

(51) Int. Cl. A61K 8/02; A61Q 5/08

(52) U.S. Cl. CPC A61K 8/0233; A61Q 5/08
USPC 132/208; 156/193; 427/177; 424/401; 424/62

(57) ABSTRACT

The invention relates to a laminate that has at least two layers, in which at least one layer of the laminate is a water-soluble polymer film containing active substances (=“water-soluble polymer film containing active substances”), the polymer forming the film is a water-soluble polymer, and at least one second layer of the laminate is a nonwoven fabric, knitted fabric, woven fabric or a foam film (=“water retention layer”) consisting of a water-soluble polymer. The water-soluble polymers of the two layers are identical or different. The invention further relates to the production of such a laminate and to the use thereof.

The invention relates to a laminate of at least one active-ingredient-containing, water-soluble polymer film (=“active-ingredient-containing, water-soluble polymer layer”) and a flat support with water absorption capacity (=“water retention layer”).

Active-ingredient-containing, water-soluble polymer films are known (e.g. from EP-A-0 450 141, WO 98/026764, WO 00/018365, US-A-2001/006677, WO 02/002085, WO 04/060298 or WO 05/009386). To produce such films, active ingredients are dissolved or dispersed in a polymer or a polymer solution and this solution or dispersion is molded to give films with or without the help of a flat support, then dried and separated from any support used and made into saleable products. Known polymer films of this type are e.g. oral hygiene strips (Listerine dental bleaching strips (Crest Whitestrips) or pharmaceuticals, e.g. to combat coughs, sneezing, chills (Theraflu® Cold/Cough Nighttime Strips, Triaminic Thin Strips®). The spectrum of active ingredients that can be used in such films is large and ranges from inorganic solids such as peroxides to complex organic compounds (pharmaceuticals) and natural substances.

Provided the active ingredients dissolve readily and rapidly in water and are able to directly develop their activity following dissolution in aqueous media, the described technology can be used advantageously. However, problems arise if although the contact with water is adequate in terms of amount, the dissolution rate of the polymer or active ingredient does not suffice in terms of time to completely dissolve (the polymer and) the active ingredient (e.g. if the strips are only briefly doused with water). For known active-ingredient-containing, water-soluble polymer films, the combination of active ingredient and polymer is unable to store or make available for a sufficiently long time an amount of water adequate for total dissolution.

Further complications arise when the active ingredient (in dissolved or solid form) still has to be activated prior to application for example (e.g. carbonates for producing carbon dioxide, for example for foaming purposes), especially if this activation cannot be left to the user without risk, but has to already be provided in the ready-to-use active-ingredient-containing polymer film in some form. Examples thereof are: persulfates (for example for bleaching hair), which have to be activated with carbamide peroxide or hydrogen peroxide. Here, it cannot be left to the user to spray the polymer-film-bonded persulfate for example with a carbamide peroxide solution prior to application; the risk of injury to a user unaccustomed to handling these substances would be too great (eye injuries, mucosa irritations, skin irritations). In such a case, the activator should expediently already be provided quasi in “kit form”, meaning that it only has to be triggered by the user.

The object was therefore to provide an
active-ingredient-containing, water-soluble polymer film which is able
   to store and/or
   provide for an adequate length of time
an amount of water sufficient at least to dissolve the active ingredient
and optionally
comprises an activator for the active ingredient, where
if necessary, the activator is prevented from reacting prematurely with the active ingredient.

This object is achieved by an at least two-layered laminate, where
at least one layer of the laminate is an active-ingredient-containing, water-soluble polymer film (1) (=“active-ingredient-containing, water-soluble polymer layer”) and
at least one second layer of the laminate is a nonwoven fabric, knitted fabric, woven fabric or a foam film which consists of a water-soluble polymer (=“water retention layer” (2)).

### DEFINITIONS

### Laminate

A “laminate” is understood as meaning the composite of two or more flat, i.e. two-dimensional, films (also referred to as “layer” above and below) with only a small thickness, usually in each case 10 to 5000 μm (preferably 50 to 1000 μm, particularly preferably 100 to 800 μm), the composite of the films being provided such that it does not delaminate under product-typical treatment (e.g. upon bending or opening) (delamination force >5 N/mm in the 180° T-peel test [15 mm laminate width, 1 cm/s peeling speed]). Such laminates are obtained e.g. by coextrusion through a multilayer nozzle, by bringing together two or more pre-produced film webs with or without adhesion promoters (e.g. adhesives) between two or more webs or by means of coating, for example extrusion coating on one or both surfaces of a pre-produced film web, or by means of any desired combinations of the described methods.

### Layer

A layer is a film-like constituent of the laminate according to the invention (see definition: laminate, above).

### Active Ingredient

In the present invention, an active ingredient is understood as meaning a substance which causes a cosmetic or pharmaceutical effect in people or animals or which, as a consequence of a chemical reaction, brings about a chemical or physical change in itself (e.g. release of a gas) or the medium in contact with the active ingredient (e.g. skin, dental enamel, hair) (e.g. pH change, oxidation).

Examples thereof are: surfactants, detergents, skincare agents, bleaches, pH regulators, food supplements, but also pharmaceutical active ingredients.

### Activator

The activator likewise belongs to the category of active ingredients, but is tailored in its function to the active ingredient “to be activated” and enables said active ingredient to develop its activity. Examples of “active ingredient-activator pairs” are: carbonates-(carboxylic) acids, peroxides-water, polymer-crosslinkers, monomer-starters, enzyme-prodrug, etc.

### Water-Soluble Polymer Film

According to the invention, water-soluble polymer films are understood as meaning films of film-forming water-soluble polymers (e.g. PVA, PVP, polysaccharides, etc.) and optionally auxiliaries such as plasticizers, surface-active substances, dyes, fragrances, sweeteners, flavorings, flavor improvers, disintegrants, etc.

Such water-soluble polymer films and their production are described for example in DE-A-196 46 392, to which reference is expressly made at this point.

“Water-soluble polymers” according to the invention dissolve in water to at least 90% by weight, preferably 95% by weight (measurement conditions: 10 g of polymer in particle form [d₅₀: between 500 and 5000 μm] in 200 ml of H₂O, 25° C. with stirring [200-1000 rpm], 30 min, filtering through white band filters (filter group 2), determination of the residue by differential weighing). Any residue present is usually undissolved, swollen polymer.

### Nonwoven Fabric, Knitted Fabric, Woven Fabric, Foam Film

Nonwoven Fabrics, Knitted Fabrics and woven fabrics are produced—by processes known to the person skilled in the art—from polymer fibers, where the fiber polymer is a water-soluble polymer (e.g. PVA, PVP, polysaccharides, etc.). The foam films of the present invention are likewise produced from water-soluble polymers, the procedure usually involving firstly dissolving the polymer in water and then introducing a gas into the polymer solution, whereupon the solution froths up. The air-bubble-containing mass (the foam) is then spread onto a suitable substrate as film or layer and subsequently dried. As a result of solvent removal, the foam solidifies during drying to give an aerogel, with the cavities formed being given a permanent structure. Such foams and their production are described for example in DE-A-100 32 456, to which reference is expressly made at this point.

### DETAILED DESCRIPTION

### Active-Ingredient-Containing, Water-Soluble Polymer Film

The active-ingredient-containing, water-soluble polymer film of the present invention comprises, as essential constituents, a water-soluble polymer or a combination of water-soluble polymers and an active ingredient and optionally one or more plasticizers and/or surface-active substances. Such films are described for example in DE-A-196 46 392, to which reference is expressly made at this point.

The polymers used for the film include hydrophilic and/or water-soluble polymers such as cellulose derivatives, hydroxypropylmethylcellulose, hydroxyethylcellulose, hydroxypropylcellulose, carboxymethylcellulose, polyvinylpyrrolidone, polyvinyl alcohol, sodium alginate, polyethylene glycol, natural gums such as xanthan resin, tragacanth, guar resin, acacia resin, gum arabic, water-dispersible polyacrylates such as polyacrylic acid, methyl methacrylate copolymers, carboxyvinyl copolymers. Preferred polymers are polyvinylpyrrolidone and polyvinyl alcohol.

The concentration of the water-soluble polymer in the finished film is typically between 2 and 70% by weight, preferably between 5 and 50% by weight. A particularly preferred concentration is ca. 20% by weight.

The interface-active substances (=surfactants) optionally used for the water-soluble polymer film can include one or more nonionic surface-active substances. Here, preference is given to those interface-active substances which have an HLB value of 5.0 and greater. For the definition of the HLB value, reference is made expressly to the statements in Hugo Janistyn, Handbuch der Kosmetika und Riechstoffe [Handbook of cosmetics and fragrances], 3rd volume: Die Körperpflegemittel [Body care compositions], 2nd edition, Dr. Alfred Hüthig Verlag Heidelberg, 1973, pages 68-78, and Hugo Janistyn, Taschenbuch der modernen ParfUmerie und Kosmetik [Pocketbook of modern perfumery and cosmetics], 4th edition, Wissenschaftliche Verlagsgesellschaft m.b.H. Stuttgart, 1974, pages 466-474, and also the original papers cited therein.

Particularly preferred nonionogenic surface-active substances here are, on account of the simple processability, substances which are commercially available in pure form as solids or liquids. In this connection, the definition of purity does not refer to chemically pure compounds. Instead, particularly if they are natural-based products, mixtures of different homologs can be used, for example with various alkyl chain lengths, as are obtained for products based on natural fats and oils. Also in the case of alkoxylated products, usually mixtures of different degrees of alkoxylation are present. In this connection, the term purity refers rather to the fact that the selected substances should preferably be free from solvents, extenders and other concomitants.

Preferred nonionogenic interface-active substances are:
Alkoxylated fatty alcohols having 8 to 22, in particular 10 to 16, carbon atoms in the fatty alkyl group and 1 to 30, in particular 1 to 15, ethylene oxide and/or propylene oxide units. Preferred fatty alkyl groups are for example lauryl, myristyl, cetyl, but also stearyl, isostearyl and oleyl groups. Particularly preferred compounds of this class are for example lauryl alcohol with 2 to 4 ethylene oxide units, oleyl and cetyl alcohol having in each case 5 to 10 ethylene oxide units, cetyl and stearyl alcohol, and mixtures thereof with 10 to 30 ethylene oxide units, and also the commercial product Aethoxal®B (Henkel), a lauryl alcohol having in each case 5 ethylene oxide and propylene oxide units. Besides the customary alkoxylated fatty alcohols, it is also possible to use so-called “terminally capped” compounds in accordance with the invention. In these compounds, the alkoxy group at the end has no OH group, but is “capped” in the form of an ether, in particular a C₁-C₄-alkyl ether. One example of such a compound is the commercial product Dehypon® LT 054, a C₁₂₋₁₈-fatty alcohol+4.5 ethylene oxide butyl ether.
Alkoxylated fatty acids having 8 to 22, in particular 10 to 16, carbon atoms in the fatty acid group and 1 to 30, in particular 1 to 15, ethylene oxide and/or propylene oxide units. Preferred fatty acids are for example lauric acid, myristic acid, palmitic acid, stearic acid, isostearic acid and oleic acid.
Alkoxylated, preferably propoxylated and in particular ethoxylated, mono-, di- and triglycerides. Examples of preferred compounds are glycerol monolaurate+20 ethylene oxide and glycerol monostearate+20 ethylene oxide.
Polyglycerol esters and alkoxylated polyglycerol esters.

Preferred compounds of this class are, for example, poly(3)glycerol diisostearate (commercial product: Lameform®TGI (Henkel)) and poly(2)glycerol polyhydroxystearate (commercial product: Dehymuls®PGPH (Henkel)).
Sorbitan fatty acid esters and alkoxylated sorbitan fatty acid esters such as, for example, sorbitan monolaurate and sorbitan monolaurate+20 ethylene oxide (EO).
Alkylphenols and alkylphenol alkoxylates with 6 to 21, in particular 6 to 15, carbon atoms in the alkyl chain and 0 to 30 ethylene oxide and/or propylene oxide units. Preferred representatives of this class are, for example, nonylphenol+4 EO, nonylphenol+9 EO, octylphenol+3 EO and octylphenol+8 EO.

Particularly preferred classes of nonionogenic interface-active substances are the alkoxylated fatty alcohols, the alkoxylated fatty acids and also the alkylphenols and alkylphenol alkoxylates.

The total concentration of the interface-active substances in the finished film depends on the properties of the other ingredients, but should expediently be between 0.1 and 5% by weight.

Plasticizers which can be used according to the invention are in particular polyalcohols. Examples of polyalcohols include glycerol, polyethylene glycol, propylene glycol, glycerol monoesters with fatty acids or other pharmaceutically used polyalcohols and moreover dexpanthenol, a trihydroxyamide. The concentration of any plasticizer in the dry mass of the film is usually 0.1 to 15% by weight.

Therapeutic active ingredients that can be used are for example: hypnotics, sedatives, antiepileptics, amphetamines, psychoneurotropics, neuro-muscle blockers, antispasmodics, antihistamines, antiallergics, cardiotonics, antiarrhythmics, diuretics, hypotensives, vasopressors, antitussives, expectorants, thyroid hormones, sex hormones, antidiabetics, antitumor active ingredients, antibiotics, and chemotherapeutics and narcotics. The amount of active ingredient to be incorporated in the film depends on its type and is usually between 0.01 and 20% by weight, but it can be higher or lower as necessary for achieving the desired effect.

Cosmetic active ingredients include skincare agents, such as dexpanthenol, haircare compositions such as shampoos, breath fresheners such as menthol, other flavorings, aroma substances or fragrances, as are customarily used for oral hygiene, and/or active ingredients for dental care and/or oral hygiene, for example quaternary ammonium bases. The effect of flavorings and aroma substances can be enhanced by flavor enhancers such as tartaric acid, citric acid, vanillin or the like. Further cosmetic active ingredients are bleaches such as carbamide peroxide, as are used in dental hygiene, or persulfates for bleaching hair.

As a further additive, the active-ingredient-containing, water-soluble polymer film can comprise one or more alkalizing agents, optionally in the form of coated particles.

The alkalizing agent serves to establish an alkaline pH. According to the invention, alkalizing agents such as ammonium, alkali metal and alkaline earth metal hydroxides, carbonates, hydrogencarbonates, silicates, in particular metasilicates, and also alkali metal phosphates can be used. Short-chain amines such as monoethanolamines, 3-amino-2-methylpropanol or alkaline reacting amino acids such as arginine, ornithine and lysine can also be used.

In one preferred embodiment, the particulate alkalizing agents comprise particles with a particle core made of solid alkalizing agents, which are selected from alkali metal carbonates, phosphates and/or silicates. Alkalizing agents that are particularly preferred according to the invention are alkali metal silicates, in particular metasilicates.

In a further preferred embodiment, the active-ingredient-containing, water-soluble polymer films according to the invention comprise at least two different alkalizing agents. In this connection, mixtures for example of a metasilicate and a hydroxycarbonate may be preferred.

The active-ingredient-containing, water-soluble polymer films according to the invention comprise alkalizing agents (calculated as uncoated alkalizing agent) preferably in amounts of from 1 to 50% by weight, in particular 15 to 35% by weight, in each case based on the layer of active-ingredient-containing, water-soluble polymer film.

In a preferred embodiment of the invention, the laminate according to the invention is used as bleaching foil for bleaching hair. In this case, one layer of the laminate, preferably the active-ingredient-containing, water-soluble polymer film, comprises a bleaching agent. Bleaching agents preferably comprise a peroxo compound. The choice of this peroxo compound is not in principle subject to any restrictions. Preferred peroxo compounds are hydrogen peroxide (H₂O₂), for example in the form of an aqueous solution or in the form of an H₂O₂ adduct onto solid supports, in which case urea perhydrate and sodium carbonate peroxohydrate (“sodium percarbonate”) have particular significance. In addition to hydrogen peroxide or instead of it, other peroxo compounds may also be present in laminate layers.

Customary peroxo compounds known to the person skilled in the art are, for example, ammonium peroxodisulfate, potassium peroxodisulfate, sodium peroxodisulfate, ammonium persulfate, potassium persulfate, sodium persulfate, potassium peroxidiphosphate, percarbonates such as magnesium percarbonate, peroxides such as barium peroxide, and also perborates, urea peroxide and melamine peroxide. Among these peroxo compounds, which can also be used in combination, preference is given according to the invention to the inorganic compounds. Particular preference is given to the peroxodisulfates, in particular combinations of at least two peroxodisulfates.

Preferred laminate layers are therefore characterized in that they additionally comprise a solid peroxo compound which is preferably selected from hydrogen peroxide addition compounds onto solid supports, ammonium and alkali metal persulfates and peroxodisulfates, with particularly preferred layers comprising at least two different peroxodisulfates.

The peroxo compounds are present in the laminate layer according to the invention, in particular the active-ingredient-containing, water-soluble polymer film, preferably in amounts of from 2 to 80% by weight, in particular in amounts of 20 to 45% by weight.

Furthermore, the aforementioned bleaching compositions can comprise further active ingredients, auxiliaries and additives such as, for example,
nonionic polymers such as, for example, vinyl-pyrrolidinone/vinyl acrylate copolymers, polyvinylpyrrolidinone and vinylpyrrolidinone/vinyl acetate copolymers and polysiloxanes,
cationic polymers such as quaternized cellulose ethers, polysiloxanes with quaternary groups, dimethyldiallylammonium chloride polymers, acrylamide-dimethyldiallylammonium chloride copolymers, dimethylaminoethyl methacrylate-vinylpyrrolidone copolymers quaternized with diethyl sulfate, vinylpyrrolidinone-imidazolinium methochloride copolymers and quaternized polyvinyl alcohol,
zwitterionic and amphoteric polymers such as, for example, acrylamidopropyltrimethylammonium chloride/acrylate copolymers and octylacrylamide/methyl methacrylate/tert-butylaminoethyl methacrylate/2-hydroxypropyl methacrylate copolymers,
anionic polymers such as, for example, polyacrylic acids, crosslinked polyacrylic acids, vinyl acetate/crotonic acid copolymers, vinylpyrrolidinone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,
thickeners such as agar agar, guar gum, alginates, xanthan gum, gum arabic, karaya gum, carob seed flour, linseed gums, dextrans, cellulose derivatives, e.g. methylcellulose, hydroxyalkylcellulose and carboxymethylcellulose, starch fractions and derivatives such as amylose, amylopectin and dextrins, clays such as e.g. bentonite or fully synthetic hydrocolloids such as e.g. polyvinyl alcohol,
structurants such as glucose, maleic acid and lactic acid,
hair-conditioning compounds such as phospholipids, for example soybean lecithin, egg lecithin and cephalins, and also silicone oils,
protein hydrolysates, in particular elastin, collagen, keratin, milk protein, soybean protein and wheat protein hydrolysates, their condensation products with fatty acids, and quaternized protein hydrolysates,
perfume oils, dimethyl isosorbide and cyclodextrins,
solvents and solubility promoters such as ethanol, isopropanol, ethylene glycol, propylene glycol, glycerol and diethylene glycol,
fiber-structure-improving active ingredients, in particular mono-, di- and oligosaccharides such as, for example, glucose, galactose, fructose, fruit sugar and lactose,
active ingredients such as panthenol, pantothenic acid, allantoin, pyrrolidonecarboxylic acids and salts thereof,
quaternized amines such as methyl-1-alkylamidoethyl-2-alkylimidazolinium methosulfate
antifoams such as silicones,
dyes for coloring the composition,
antidandruff active ingredients such as piroctone olamine, zinc omadine and climbazole,
photoprotective agents, in particular derivatized benzophenones, cinnamic acid derivatives and triazines,
substances for adjusting the pH such as, for example, customary acids, in particular food acids and bases,
active ingredients such as allantoin, pyrrolidone-carboxylic acids and salts thereof, and bisabolol,
vitamins, provitamins and vitamin precursors, in particular those of groups A, B3, B5, B6, C, E, F and H,
plant extracts such as the extracts from green tea, oak bark, stinging nettle, hamamelis, hops, chamomile, burdock, horsetail, hawthorn, linden blossom, almond, Aloe Vera, fir needle, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, Melissa, rest-harrow, coltsfoot, marshmallow, meristem, ginseng and ginger root,
cholesterol,
consistency regulators such as sugar esters, polyol esters or polyol alkyl ethers,
fats and waxes such as spermaceti, beeswax, montan wax and paraffins, fatty alcohols and fatty acid esters,
fatty acid alkanolamides,
complexing agents such as EDTA, NTA, β-alaninediacetic acid and phosphonic acids,
swelling and penetration substances such as glycerol, propylene glycol monoethyl ether, carbonates, hydrogencarbonates, guanidines, ureas, and primary, secondary and tertiary phosphates,
opacifiers such as latex, styrene/PVP and styrene/acrylamide copolymers,
pearlizing agents such as ethylene glycol mono- and distearate, and also PEG-3 distearate, pigments,
stabilizers for hydrogen peroxide and other oxidizing agents,
propellants such as propane/butane mixtures, N₂O, dimethyl ether, CO₂ and air,
antioxidants,
dyes,
fragrances and
disintegrants.

The specified substances, which can be mixed into the film as desired, must be safe as regards toxicity and should be approved for use in cosmetics, medicinal products and pharmaceutical products by the relevant authorities.

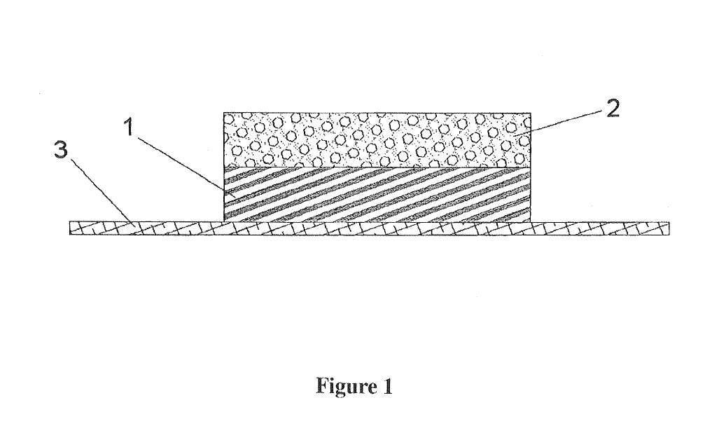

FIG. 1 shows the structure of the laminate according to the invention made of the active-ingredient-containing, water-soluble polymer layer (1), the water retention layer (2) and the optionally present support layer (3).

The active-ingredient-containing, water-soluble film (1) can be prepared as follows:

The active ingredient and the optionally used surface-active substances, the optionally used plasticizers, disintegrants and other possible constituents apart from the water-soluble polymer or polymers are dissolved or dispersed with a sufficient amount of a compatible solvent. Examples of a compatible solvent include water, alcohols such as ethyl alcohol, esters such as ethyl acetate, ketones such as acetone, ethers such as diethyl ether, tetrahydrofuran (THF), dimethylformamide (DMF), dimethyl sulfoxide DMSO or mixtures thereof. After forming a solution or suspension, the water-soluble polymer or the mixture of water-soluble polymers is added slowly with stirring and, if necessary, heat, until a homogeneous solution or a homogeneous pasty mass has formed. Alternatively, and provided the starting materials used allow this, the mass can also be prepared without using solvents.

The resulting mass is then expediently applied to a support (3) and dried to give a film. The support material must have a surface tension which enables the polymer solution or mass to spread uniformly over the intended coating width without the solution/mass soaking in and thus a destructive bond between support and coating being formed. Examples of suitable materials include metal films such as aluminum films, non-siliconized polyethylene terephthalate films, non-siliconized kraft paper, polyethylene-impregnated kraft paper or non-siliconized polyethylene film.

The solution or mass can be applied to the support material (3) using any customary apparatus. Specifically preferred application technology relates to a knife-over-roll coating machine. The thickness of the resulting film layer depends on the concentration of the solids in the coating solution or mass and also on the gap width of the coating machine and can vary between 5 and 5000 μm. The drying of the film is carried out e.g. in a hot-air bath using a drying furnace, drying tunnel, vacuum dryer or other suitable drying apparatuses which do not adversely impair the effect of the active ingredient. Then, the film—including support film or after separation from the support film—is further processed or, for better ease of use—is directly cut or made into sections of suitable size and shape.

### Nonwoven Fabric, Knitted Fabric, Woven Fabric, Foam Film (=Water Retention Layer (2))

The second layer (2) of the laminate according to the invention consists of a water-soluble polymer, the layer made of this polymer being configured such that it has an increased water retention capacity. This can take place for example by constructing the layer from staple fibers of the water-soluble polymer in the form of a nonwoven fabric, or corresponding fibers of the water-soluble polymer being processed to give corresponding knitted fabrics or woven fabrics.

The thicknesses of the nonwoven fabrics, knitted fabrics or woven fabrics used according to the invention vary between 5 and 1000 μm, preferably 10 to 500 μm and particularly preferably 20 to 200 μm. They expediently have areal weights of from 5 to 1000 g/m², preferably 10 to 500 g/m², particularly preferably 20 to 200 g/m².

According to the invention, preference is given to nonwoven fabrics, in particular made of PVA, as are sold for example by Freudenberg (Weinheim, Germany) under the name Vilene (DO 101 or DO 102).

A particularly advantageous configuration of the water retention layer (2) is a foam film made of water-soluble polymer, as is described for example in DE-A-100 32 456, to which reference is expressly made at this point. Such foams disintegrate or dissolve rapidly in an aqueous environment and, moreover, have the advantage that (further) active ingredients (e.g. an activator) can be provided for release in the cavities of the foam which, on account of their encapsulation in the foam, have no contact with materials outside of the polymer shell of the foam surrounding them on all sides.

The spaces or cavities of the foam can accordingly be filled with a gas, a gas mixture, a liquid or a liquid mixture. The polymer of the matrix is a water-soluble polymer, as is also used for the water-soluble films described above, although a polyvinyl alcohol-polyethylene glycol graft copolymer is preferably used.

The spaces or cavities of the foam film (2) can in each case be present in the polymer matrix isolated from one another, preferably in the form of solidified bubbles (as are known for example for PU or PS foams).

The gas or gas mixture in the cavities is preferably air, nitrogen, oxygen, carbon dioxide, helium or any desired mixture of these gases, particularly preferably air. However, it may also be advantageous if the spaces or cavities are filled with a liquid or a liquid mixture (for example an oil), where these liquids are not miscible with the matrix material and do not dissolve the polymer backbone of the matrix. The liquid or the liquid mixture can moreover comprise one or more active ingredients.

The thickness of the foam film is preferably 0.1 to 5 mm, particularly preferably 0.5 to 1 mm.

To produce the foam film (2), for example, firstly a solution or dispersion is prepared which comprises the water-soluble polymer and optionally an active ingredient. This solution, which may also be a concentrated solution or viscous mass, is then frothed up by introducing a gas or gas mixture (e.g. air). This can be carried out by means of a disperser or a foam beating machine, but also by means of other methods, e.g. by means of ultrasound, but preferably with the help of a foam beating machine. As a result, the diameter of the bubbles can be adjusted within a wide range, almost arbitrarily. For example, the diameter of the bubbles or cavities can be in the range from 0.01 to 50 μm; preference is given to bubbles/cavities with a diameter between 0.1 and 10 μm. In order to stabilize the foams or air-bubble-containing (or gas-bubble-containing or active-ingredient-containing) masses thus produced, a foam-stabilizing agent can be added before or during the generation of the foam. Agents suitable for this purpose, e.g. surfactants, are known to the person skilled in the art and are listed by way of example above. Finally, the foam is spread onto the active-ingredient-containing, water-soluble polymer layer, or if the foam film is prepared separately, onto a suitable substrate as film or layer and subsequently dried. As a result of solvent removal, the foam solidifies during drying to give an aerogel, with the cavities formed being given a permanent structure.

The shape, number and size of the spaces and cavities produced can be influenced by means of various process parameters, e.g. through the concentration of the polymer, through the viscosity of the polymer mass, by controlling the foaming process (see above) or through selection of the foam-stabilizing agents.

Further alternative processes for producing the foam film are described in DE-A-100 32 456.

### Water-Soluble Laminate

The laminate according to the invention is produced from the active-ingredient-containing polymer film and the water-soluble nonwoven fabric, knitted fabric, woven fabric or the water-soluble foam film.

Expediently, the procedure here involves separately preparing the active-ingredient-containing polymer film and the water-soluble nonwoven fabric, knitted fabric, woven fabric or the water-soluble foam film, superimposing them and then bringing them together and rolling them up or assembling them to give the laminate according to the invention.

Alternatively, in the preparation process of the active-ingredient-containing polymer film, the water-soluble nonwoven fabric, knitted fabric, woven fabric or the water-soluble foam film can also be laminated onto this—for example before or after drying—or spread on in the form of a mass (or vice versa: in the production process of the water-soluble nonwoven fabric, knitted fabric, woven fabric or the water-soluble foam film, the active-ingredient-containing polymer film is laminated or spread onto these). Then, the laminate—including support film or after separation from the support film—is rolled up and stored or cut into sections of suitable size and shape and packaged.

The advantage of the laminate according to the invention is that the laminate, following the addition of water, becomes completely soaked similar to a sponge, and/or a relatively large amount remains on the material surface. Here, firstly the thin water-soluble covering (2) (for example the nonwoven fabric) is dissolved, before relatively large amounts of water are available for the subsequent active ingredient release.

The laminates according to the invention are exceptionally suited to the bleaching of hair. In this connection, the procedure is generally as follows:
wetting the water retention layer of the laminate according to the invention with an aqueous medium, preferably with an aqueous, 2-10% strength by weight H₂O₂ solution or a carbamide peroxide gel; (directly) afterwards
placing a hair tress on the wetted water retention layer,
optionally covering the hair tress with a flat covering, which is impermeable to active ingredients and optionally additives,
releasing the active ingredients by virtue of prolonged contact between the wetted water retention layer and the active-ingredient-containing layer,
allowing the active ingredients to act on the hair tress for a period of at least 5 min, preferably 20 to 120 min, during which bleaching takes place; the “allowing to act” can take place with the additional introduction of heat, as a result of which the contact time can be shortened and/or the bleaching can be intensified,
removing the laminate and any sheet-like covering present,
optionally rinsing the hair tress with water.

The invention is explained in more detail below by reference to examples.

### Example 1

With vigorous stirring, 25 g of polyvinylpyrrolidone polymer (PVPP) (Kollidon® 30, BASF) and 5 g of disintegrant (Kollidon® CL-SF, BASF) were dissolved in 30 g of ethanol and 6 g of water. 70 g of carbamide peroxide were added in portions and the mixture was stirred to the point of optical homogeneity. The pH was then adjusted to 4 through the dropwise addition of phosphoric acid and testing by means of a pH electrode.

The resulting paste was applied to a support film (aluminum film, 40 g/m², Korff, Switzerland) using a manual coating bar (gap width 1000 μm), covered with a water-soluble nonwoven fabric (VILENE® DO 101 from Freudenberg, Weinheim, Germany) and then left to stand for 24 hours at room temperature such that a dry laminate with an areal weight of ca. 420 g/m² resulted (circular stamping press 50 cm², weighing by means of laboratory analytical balance).

The resulting product was separated into sections each measuring 10.17 cm and packaged individually into four-edge sealed pouches. The packaging used was a composite made of paper/Alu/PE.

### Example 2

With vigorous stirring, firstly 20 g of PVPP (Kollidon 30) and then 5 g of disintegrant (Kollidon CL-SF) were added to 35 g of ethanol. 10 g of plasticizer (dexpanthenol 100%) were introduced into the homogeneous mass and stirred until completely dissolved. In portions, 65 g of a solid mixture consisting of components for bleaching hair (alkali metal silicate (peroxide stabilizer); sodium persulfate (bleach); ammonium persulfate (bleach); disodium EDTA (complexing agent, stabilizer), silica) were added and the mixture was stirred to the point of optical homogeneity.

The resulting paste was applied to a support film by means of a manual coating bar (gap width 600 μm), covered with a water-soluble nonwoven fabric (VILENE DO 101 from Freudenberg, Weinheim, Germany) and then left to stand for 24 hours at room temperature such that a dry laminate with an areal weight of ca. 520 g/m² resulted (circular stamping press 50 cm², weighing by means of laboratory analytical balance).

The resulting product was separated into sections each measuring 10·17 cm and packaged individually into four-edge sealed pouches. The packaging used was a composite of paper/Alu/PE.

### Example 3

With vigorous stirring, firstly 17 g of PVPP (Kollidon 30) and then 3 g of disintegrant (Kollidon CL-SF) were added to 35 g of ethanol. 12 g of plasticizer (triethyl citrate) were introduced into the homogeneous mass and stirred until completely dissolved. In portions, 70 g of a solid mixture consisting of components for bleaching hair (persulfates, silicates, etc., see example 2) were added and stirred to the point of optical homogeneity.

The resulting paste was applied to a support film by means of a manual coating bar (gap width 600 μm), covered with a water-soluble nonwoven fabric (VILENE DO 101 from Freudenberg, Weinheim, Germany) and then left to stand for 24 hours at room temperature such that a dry laminate with an areal weight of ca. 490 g/m² resulted (circular stamping press 50 cm², weighing by means of laboratory analytical balance).

The resulting product was separated into sections each measuring 10·17 cm and packaged individually into four-edge sealed pouches. The packaging used was a composite of paper/Alu/PE.

### Example 4 (Application Example)

The laminates from examples 1 to 3 were used for bleaching hair. For this, the procedure was as follows:

The water retention layer of the laminates was treated with an aqueous hydrogen peroxide solution or gel (4-9% by weight). Then, in each case, one hair tress was placed onto the wetted water retention layer. Wrapping the laminate around the tress resulted in close contact between the tress and the active ingredients. After a contact time of 45 min without the external introduction of heat, the laminates were removed and the tresses were rinsed with water.

### Description of the Results:

The hair treated in this way exhibited different degrees of color lightening depending on laminate thickness and applied mass of hydrogen peroxide.

1. A two-layered laminate comprising two or more films or layers, where
one layer of the laminate is an active-ingredient-containing, water-soluble polymer film or active-ingredient-containing, water-soluble polymer layer, where the polymer forming the film or layer is a water-soluble polymer, and
a second layer of the laminate is a nonwoven fabric, knitted fabric, woven fabric or a foam film or water retention layer of a water-soluble polymer, where the water-soluble polymers of the two layers are identical or different,
wherein the active-ingredient-containing, water-soluble polymer film comprises at least one peroxo compound in amounts of from 20 to 45% by weight.

2. The laminate as claimed in claim 1, wherein the water-soluble polymer is selected from: cellulose derivatives, polyvinylpyrrolidone, polyvinyl alcohol, sodium alginate, polyethylene glycol, natural gums, water-dispersible polyacrylates and mixtures thereof.

3. The laminate as claimed in claim 1, wherein the water-soluble polymer is present in the active-ingredient-containing, water-soluble polymer film in a concentration of between 2 and 70% by weight.

4. The laminate as claimed in claim 1, wherein the active-ingredient-containing, water-soluble polymer film comprises interface-active substances or surfactants.

5. The laminate as claimed in claim 1, wherein the active-ingredient-containing, water-soluble polymer film comprises plasticizers.

6. The laminate as claimed in claim 1, wherein the active-ingredient-containing, water-soluble polymer film comprises one or more alkalizing agents optionally in the form of coated particles.

7. The laminate as claimed in claim 1, wherein the second layer of the laminate is composed of staple fibers of a water-soluble polymer in the form of a nonwoven fabric, or is composed of corresponding fibers of a water-soluble polymer in the form of a knitted fabric or woven fabric.

8. The laminate as claimed in of claim 1, wherein the second layer of the laminate is a foam film of a water-soluble polymer.

9. The laminate as claimed in claim 1, wherein the laminate comprises a third layer or support.

10. A process for producing a laminate as claimed in claim 1, comprising
producing the active-ingredient-containing polymer film and the water-soluble nonwoven fabric, knitted fabric, woven fabric or the water-soluble foam film separately superimposing and then bringing together and rolling or assembling to give the laminate
or laminating the water-soluble nonwoven fabric, knitted fabric, woven fabric or the water-soluble foam film onto the polymer film or spreading on as a mass during the production process of the active-ingredient-containing polymer film, optionally with the help of a support film, and then rolling up the laminate, including any support film or after separating off any support film, and storing or cutting the laminate into sections of suitable size and shape and packaging;
or laminating or spreading on the active-ingredient-containing polymer film onto the water-soluble nonwoven fabric, knitted fabric, woven fabric or the water-soluble foam film during the production process of the water-soluble nonwoven fabric, knitted fabric, woven fabric or the water-soluble foam film, optionally with the help of a support film, and then rolling up the laminate, including any support film or after removing any support film, and storing or cutting the laminate into sections of suitable size and shape and packaging.

11. Hair bleach comprising a laminate as claimed in claim 1.

12. A method for bleaching hair comprising the steps:
wetting the water retention layer of a laminate as claimed in claim 1 with an aqueous medium,
placing a hair tress on the wetted water retention layer,
releasing the active ingredients by virtue of prolonged contact between the wetted water retention layer and the active-ingredient-containing layer,
allowing the active ingredients to act on the hair tress and
removing the laminate.

13. The laminate as claimed in claim 4, wherein the interface-active substances or surfactants are one or more nonionic or nonionogenic surface-active substances.

14. The laminate as claimed in claim 4, wherein the interface-active substances or surfactants are one or more nonionic surface-active substances having an HLB value of 5.0 and greater.

15. The laminate as claimed in claim 4, wherein the interface-active substances or surfactants comprise
alkoxylated fatty alcohols having 8 to 22 carbon atoms in the fatty alkyl group and 1 to 30 ethylene oxide and/or propylene oxide units and/or
alkoxylated fatty acids having 8 to 22 carbon atoms in the fatty acid group and 1 to 30 ethylene oxide and/or propylene oxide units and/or
alkoxylated mono-, di- and triglycerides and/or
polyglycerol esters and alkoxylated polyglycerol esters and/or
sorbitan fatty acid esters and alkoxylated sorbitan fatty acid esters and/or
alkylphenols and alkylphenol alkoxylates having 6 to 21 carbon atoms in the alkyl chain and 0 to 30 ethylene oxide and/or propylene oxide units.

16. The laminate as claimed in claim 4, wherein the interface-active substances or surfactants are present in a total concentration of between 0.1 and 5% by weight in the active-ingredient-containing, water-soluble polymer film.

17. The laminate as claimed in claim 5, wherein the plasticizers are polyalcohols.

18. The laminate as claimed in claim 5, wherein plasticizers are glycerol, dexpanthenol, polyethylene glycol, propylene glycol and/or glycerol monoesters with fatty acids.

19. The laminate as claimed in claim 5, wherein the plasticizers are present in a concentration of 0.1 to 15% by weight of the dry mass of the active-ingredient-containing, water-soluble polymer film.

20. The laminate as claimed in claim 6, wherein the alkalizing agents, calculated as uncoated alkalizing agent, are present in the active-ingredient-containing, water-soluble polymer film in amounts of from 1 to 50% by weight.

21. The laminate as claimed in claim 7, wherein the thickness of the nonwoven fabric, knitted fabric or woven fabric varies between 5 and 1000 and/or their areal weights are between 5 and 1000 g/m².

22. The laminate as claimed in claim 8, wherein the second layer of the laminate has a thickness of 0.1 to 5 mm.

23. The laminate as claimed in claim 9, wherein the laminate comprises a third layer or support, and said layer or support is selected from: metal films, non-siliconized polyethylene terephthalate films, non-siliconized kraft papers, polyethylene-impregnated kraft papers and non-siliconized polyethylene films.
